(19) United States
(12) Patent Application Publication
(10) Pub. No.: US 2026/0272871 A1
(43) Pub. Date: Sep. 17, 2026

(54) USE OF RETINOIC ACID RECEPTOR ACTIVATOR AND COMPOSITION THEREOF IN REGENERATION AND REPAIR OF MAMMALS

(71) Applicants: INSTITUTE OF ZOOLOGY, CHINESE ACADEMY OF SCIENCES, Beijing (CN); BEIJING INSTITUTE FOR STEM CELL AND REGENERATIVE MEDICINE, Beijing (CN)

(72) Inventors: Wei LI, Beijing (CN); Qi ZHOU, Beijing (CN); Zheng-Quan HE, Beijing (CN); Zongbao LU, Beijing (CN); Xuewei YUAN, Beijing (CN); Xin WANG, Beijing (CN); Shuai WANG, Beijing (CN); Liu WANG, Beijing (CN)

(73) Assignees: INSTITUTE OF ZOOLOGY, CHINESE ACADEMY OF SCIENCES, Beijing (CN); BEIJING INSTITUTE FOR STEM CELL AND REGENERATIVE MEDICINE, Beijing (CN)

(21) Appl. No.: 18/875,305

(22) PCT Filed: Jun. 16, 2023
(86) PCT No.: PCT/CN2023/100866

§ 371 Date: Dec. 16, 2024

(30) Foreign Application Priority Data
Jun. 16, 2022 (CN) 202210680476.9

(51) Int. Cl. A61K 31/203; A61K 31/192; A61K 31/197; A61K 31/45; A61K 31/4741; A61P 17/02

(52) U.S. Cl. CPC A61K 31/203; A61K 31/192; A61K 31/197; A61K 31/45; A61K 31/4741; A61P 17/02

(57) ABSTRACT

Provided is use of a retinoic acid receptor activator or a composition containing the retinoic acid receptor activator in preparing a drug or reagent for promoting the regeneration and repair capacity of tissues or complex structures or organs of mammals. The retinoic acid receptor activator comprises one or two or more of all-trans retinoic acid and a selective retinoic acid receptor y agonist. The regeneration and repair refer to promoting the regeneration of tissues or complex structures or organs after tissue or organ resection or damage.

### TECHNICAL FIELD

The present invention relates to the field of biotechnology, and in particular to a method for promoting the regeneration and repair of an organ of a mammal and related use thereof.

### BACKGROUND ART

Regeneration refers to the repair process in which a structure with the same morphology and function as the lost part grows on the basis of the remaining part after the whole body or an organ of an organism is injured and partially lost. Failure of regeneration can lead to loss of tissue or organ functionality, ultimately causing various diseases or even death. Different species in nature have different regeneration capacities, which can be divided into: 1. regeneration at the individual level, such as lower plants can regenerate a plant from a single cell, some higher plants can use roots, stems, leaves and other tissues to regenerate new plants, lower organisms such as turbellarians can use any part of the body to regenerate a complete individual; 2. regeneration after excision, tailed amphibians such as anacondas, geckos and some fish can regenerate severed tails, limbs and fins; 3. regeneration at the tissue level, such as regeneration achieved through hepatocyte proliferation after liver resection, and regeneration of skin tissue; 4. regeneration at the cell level, such as the regrowth of broken neuronal axons. However, compared with plants and lower animals, mammals, including humans, have greatly lost the capacity to regenerate and have almost no capacity to regenerate after excision. Regeneration is limited to the fetal period and specific tissues or organs, such as the liver and skin, at specific periods. The general trend is: as the evolutionary level increases, the species' regenerative capacities become weaker, or even lost.

When mammals, including humans, face various damages, scar formation occurs as a repair method, which can directly cause tissue and organ dysfunction, such as loss of movement due to amputation, or fibrosis-related diseases, such as cardiovascular disease, degenerative nervous system disease, lung disease, liver disease, diabetes, skin disease, etc. Worldwide, scar formation caused by damage to tissues and organs is the main cause of disability and death from many diseases. Therefore, improving the in situ regeneration and repair capacities of mammals is an important means to understand the essence of life and treat a series of related diseases.

In mammals, severe and chronic damages are usually repaired by scar formation rather than tissue regeneration, which is characterized by the replacement of functional tissue with a large amount of fibrotic tissue produced by extensive connective tissue hyperplasia and extracellular matrix deposition. Studies have shown that fibrosis is the main cause of disability and death from many chronic diseases. Many COVID-19 patients have post-inflammatory pulmonary fibrosis after discharge from the hospital, which greatly affects the patient's prognosis and quality of life. Currently in clinical practice, although a few drugs or cell therapy means can be used to alleviate the process of fibrosis in specific organs and specific types, they cannot restore damaged tissues. And there is an extreme lack of broad and effective anti-fibrosis means. Therefore, there is an urgent need to develop broad-spectrum anti-fibrosis and regeneration-promoting candidate targets and drugs. Fibrosis is a non-regenerative repair manner for damage. Promoting the regeneration of damaged tissue (regenerative therapy) can not only control fibrosis, but also restore the original function of the tissue. Therefore, regenerative therapy is the most ideal means to prevent and treat fibrosis-related diseases.

Pulmonary fibrosis is a pathological change characterized by the proliferation of fibroblasts and the accumulation of a large amount of extracellular matrix, accompanied by inflammatory damage and tissue structure destruction. That is, normal alveolar tissue is damaged and then repaired abnormally, resulting in structural abnormalities (scar formation). Pulmonary fibrosis can seriously affect the human respiratory function, manifesting as various breathing difficulties that worsen with the worsening of the condition and lung damage, as well as the decline of the patient's respiratory function. It is reported that the incidence and mortality of idiopathic pulmonary fibrosis are increasing year by year worldwide, and the average survival period after diagnosis is less than 3 years, which is higher than most tumors. Therefore, it is also called a “tumor-like disease.” Therefore, promoting the regeneration of damaged tissue is the most fundamental means to treat and prevent diseases related to pulmonary fibrosis and has important application value.

### SUMMARY

In order to achieve the regeneration and repair of mammalian tissues and organs and to develop more technical approaches for the prevention and treatment of related diseases, the present application provides a small molecule compound with the capacity to promote regeneration and repair, which has achieved groundbreaking and unexpected technical effects. The technical solution of this application is as follows:
The present application provides a compound that can promote the regeneration and repair capacity of a tissue and organ, and the compound is a retinoic acid receptor activator.
The present application provides use of a retinoic acid receptor activator or a composition comprising the retinoic acid receptor activator in the promotion of regeneration and repair capacity of a tissue, complex structure or organ of a mammal.
The present application also provides use of a retinoic acid receptor activator or a composition comprising the retinoic acid receptor activator in the preparation of a medicament or reagent for promoting regeneration and repair capacity of a tissue, complex structure or organ of a mammal.
The present application also provides use of a retinoic acid receptor activator or a composition comprising the retinoic acid receptor activator in the preparation of a medicament or reagent for treating a disease related to regeneration and repair of a tissue, complex structure or organ of a mammal.

Furthermore, the retinoic acid receptor activator provided in the present application may be any one or more selected from the group consisting of: an all-trans retinoic acid and a selective retinoic acid receptor y agonist.

Preferably, the selective retinoic acid receptor y agonist is CD437 and/or BMS961.

Furthermore, the regeneration and repair described in the present application is promotion of the regeneration of a tissue, complex structure or organ after tissue or organ resection or damage.

Preferably, the tissue described in the present application is skin, fat, muscle, bone, hair follicle, blood vessel or nerve.

Preferably, the complex structure described in the present application is at least any two or more of body structure comprising skin, hair follicle, gland, cartilage, muscle, fat, blood vessel, nerve, or limb.

Preferably, the organ described in the present application is lung, liver, heart, pancreatic islet or kidney.

Further preferably, the complex structure is an ear, a limb, a finger, an eye or a nose.

Preferably, the regeneration and repair described in the present application is promotion of regeneration after ear resection.

Preferably, the regeneration and repair described in the present application is promotion of regeneration and repair after skin damage, hair regeneration after hair loss, regeneration and repair of cartilage and muscle damage, regeneration of fibrosis of lung, liver, skin, heart, kidney and muscle, and regeneration after blood vessel, nerve and limb damage.

Preferably, the regeneration and repair described in the present application is promotion of the regeneration and repair of scalded skin.

Preferably, the diseases related to the regeneration and repair of the tissue or complex structure or organ described in the present application is skin scald, skin trauma, skin burn, hair loss, cartilage and muscle damage, liver fibrosis, pulmonary fibrosis or limb damage.

The retinoic acid receptor activator provided in the present application is used to promote the regeneration and repair capacity of the tissue and organ by activating retinoic acid, preferably, by activating retinoic acid receptor γ.

Furthermore, the composition comprising the retinoic acid receptor activator provided in the present application comprises a protein synthesis inhibitor, all-trans retinoic acid and a BMP activator.

Preferably, the protein synthesis inhibitor in the composition is cycloheximide (CHX), and the BMP activator is BMP signaling agonist sb4.

More preferably, in the composition, based on 1 part by weight of the cycloheximide, the amount of the all-trans retinoic acid is 0.25 to 8 parts by weight, and the amount of the BMP signaling agonist sb4 is 0.25 to 4 parts by weight.

Preferably, the protein synthesis inhibitor in the composition is Narciclasine, and the BMP activator is BMP signaling agonist sb4.

More preferably, in the composition, based on 1 part by weight of the Narciclasine, the amount of the all-trans retinoic acid is 0.25 to 10 parts by weight, and the amount of the BMP signaling agonist sb4 is 2 to 10 parts by weight.

The present application also provides that the administration manner of the retinoic acid receptor activator and the composition containing the retinoic acid receptor activator can be intraperitoneal injection, intravenous injection, oral gavage, oral administration or skin application.

The present application also provides a method for promoting regeneration and repair of a tissue, complex structure or organ of a mammal, comprising administering a retinoic acid receptor activator or a composition comprising the retinoic acid receptor activator to a subject in need thereof.

Furthermore, the retinoic acid receptor activator or the composition comprising the retinoic acid receptor activator in the method is the retinoic acid receptor activator or the composition comprising the retinoic acid receptor activator provided in the present application.

Furthermore, in the method, the retinoic acid receptor activator or the composition comprising the retinoic acid receptor activator can be administered to a subject in need thereof by intraperitoneal injection, intravenous injection, oral gavage, oral administration or skin application.

### BRIEF DESCRIPTION OF THE DRAWINGS

FIG. 1A shows the differential gene analysis and functional enrichment results between the transcriptomes of regenerative African agouti and non-regenerative mice.

FIG. 1B is a schematic diagram of the healing of a 2 mm diameter mouse ear hole after treated with Vehicle and different protein translation inhibitors Ani and CHX drugs.

FIG. 1C is a schematic diagram of the closure of a 2 mm diameter mouse ear hole after treatment with Vehicle and different doses of CHX drug.

FIG. 1D is a schematic diagram of the closure of a mouse ear hole injured by a 2 mm diameter of ear puncher after 30 days of CHX drug treatment, the scale bar is 1 mm.

FIG. 1E shows the HE staining results of the mouse auricle tissue after CHX drug treatment, and the scale bar is 200 um.

FIG. 1F shows the HE staining results of the mouse auricle tissue after CHX drug treatment, and the scale bar is 1 mm.

FIG. 1G is a schematic diagram of the KI67 immunohistochemical staining results of the mouse auricle tissue after 7 days of CHX drug treatment, and the scale bar is 100 um.

FIG. 1H is a schematic diagram of the HE staining results of the mouse auricle tissue after 180 days of the closure of the mouse ear hole treated with CHX drugs, and the scale bar is 1 mm.

FIG. 1I is a schematic diagram of the healing effect of a mouse ear hole after 3 weeks of treatment with different drug administration manners (21 days after damage).

FIG. 1J shows the healing effect of 2 mm ear holes in Nsun2 knockout mice (21 days after damage).

FIG. 2A is a schematic diagram of the closure of a 4 mm diameter mouse ear hole after treatment with DMSO/CHX.

FIG. 2B is a schematic diagram of the closure of a mouse ear hole injured by a 4 mm diameter of ear puncher after 50 days of CHX treatment, and the scale bar is 1 mm.

FIG. 3 shows the healing effect of a mouse ear hole treated with inhibitors of protein and RNA synthesis, ferroptosis and autophagy (21 days after damage).

FIG. 4 is a schematic diagram of the effect of CHX in promoting ear hole regeneration by simultaneously interfering with development and lower biological regeneration-related pathways (21 days after damage).

FIG. 5A is a schematic diagram of the effect of RAR activator, RA, in promoting ear hole regeneration (21 days after damage). RA (Retinoic acid, MCE, HY-14649, 5/20 mg/kg), activates RARs. n≥6. ***p<0.001, t-test.

FIG. 5B is a schematic diagram of the effect of RAR activator, RA, in promoting ear hole regeneration (21 days after damage). RA (Retinoic acid, MCE, HY-14649, 5/20 mg/kg), activates RARs. n≥6. ***p<0.001, t-test.

FIG. 6A is a schematic diagram of the healing of a mouse ear hole after treatment with Vehicle/CRB.

FIG. 6B is a diagram of the healing effect of a 4 mm mouse ear hole after treatment with Vehicle/CRB for 30 days.

FIG. 6C is the HE staining image of the mouse auricle tissue after treatment with Vehicle/CRB.

FIG. 6D is the immunofluorescence staining image of α-SMA of mouse auricle tissue after treatment with Vehicle/CRB for 7 days.

FIG. 6E is a schematic diagram of the HE staining results of mouse auricle tissue after the 4 mm diameter mouse ear hole was treated with Vehicle/CRB for more than 180 days, and the scale bar is 100 μm.

FIG. 6F is the immunofluorescence staining results of vascular marker CD31, and the scale bar is 20 μm.

FIG. 7 is a schematic diagram of the scar area of a scald in mice after treatment with Vehicle/RA.

FIG. 8A is a schematic diagram of limb modeling.

FIG. 8B shows the phenomenon of limb regeneration induced by drug treatment for 20, 40, and 120 days and the scale bar is 2 mm.

FIG. 8C is the statistical result of the outgrowth length of the limbs. n≥3, ***p<0.001, t-test.

FIG. 8D shows a high-definition image of the bone tissue reconstruction process obtained by electronic computed tomography (CT) after 20 days and 40 days of drug CR treatment.

FIG. 8E shows the regeneration results of bone tissue after 30 days of drug CR treatment.

FIG. 8F shows HE staining showing that the combination can promote the regeneration of multiple tissue types at the amputated limb.

FIG. 8G shows the Masson staining results of mouse limb regeneration tissue after 21, 50, and 120 days of drug treatment, and the scale bar is 2 mm, n≥3, t-test.

FIG. 9A is a schematic diagram of the healing of a 4 mm diameter mouse ear hole after treatment with Vehicle/NRB.

FIG. 9B is a schematic diagram of the immunohistochemistry results after treatment with NRB.

FIG. 9C is a schematic diagram of HE section staining results after treatment with NRB.

### DETAIL DESCRIPTION

The following is an elaboration and description of the embodiments of the present invention through specific examples, but the following contents should not be construed as limiting the present invention in any way.

The present application mainly relates to a compound capable of promoting the regeneration and repair capacity of tissues or complex structures or organs of mammals or a composition comprising the compound. Specifically, the compound is a retinoic acid receptor activator.

The present application also relates to the use of the compound in the preparation of a medicament or reagent for promoting the regeneration and repair capacity of tissues or complex structures or organs of mammals.

The present application also relates to the use of the compound in the preparation of a medicament or reagent for treating diseases related to the regeneration and repair of tissues or complex structures or organs of mammals.

Retinoic acid receptors (RARs) are nuclear receptors for retinoic acid and can be activated by retinoic acid (RA). Retinoic acid is an intermediate product of vitamin A metabolism in animals. It mainly affects bone growth and promotes epithelial cell proliferation, differentiation, keratin dissolution and other metabolic effects. The nuclear receptors of RA mainly include retinoic acid receptors (RARs) and retinoid X receptors (RXRs). RARs and RXRs act as ligand-dependent transcriptional regulatory factors in the nucleus to regulate the expression of specific target genes. RARs have three subtypes: RARα, RARβ, and RARγ, which can be activated equally. By interfering with each target factor, the results showed that activating RARα had no significant promoting effect on regeneration and repair, activating RARβ would inhibit basal level healing, while activating RARγ could promote regeneration and repair.

In a specific embodiment of the present application, the promotion of regeneration and repair capacity of the tissues and organs is achieved by activating retinoic acid or retinoic acid receptor γ. Specifically, the retinoic acid receptor activators described in the present application include compounds or combinations of compounds that can produce equal activation effects on various pathways of retinoic acid receptors (RARs), and compounds or combinations of compounds that can selectively activate the RARγ pathway in retinoic acid receptors (RARs).

In some specific embodiments, the retinoic acid receptor activator is all-trans retinoic acid, which can produce an equal activation effect on each pathway of retinoic acid receptors RARs. All-trans retinoic acid (ATRA) is one of the two active forms of retinoic acid, which is both a retinoic acid receptor (RAR) and a retinoid X receptor (RXR). RAR and RXR act as transcription factors and jointly regulate the growth and differentiation of normal and tumor cells.

In other specific embodiments, the retinoic acid receptor activator is a selective retinoic acid receptor γ agonist. Preferably, the selective retinoic acid receptor γ agonist is selected from CD437 and/or BMS961.

In the embodiments of the present application, the regeneration and repair refers to the repair process in which a structure identical in morphology and function to the lost part grows on the basis of the remaining part after the whole, organ or local tissue of a mammalian organism is traumatized and partially lost.

In a specific embodiment, the regeneration and repair is promotion of the regeneration of tissues or complex structures or organs after the resection or damage of mammalian tissues or organs.

In some specific embodiments, the tissue is epidermis, dermis, muscle, bone, fat, hair follicle, blood vessel or nerve.

In some specific embodiments, the complex structure is at least two or more of a body structure including skin, hair follicles, glands, cartilage, muscles, fat, blood vessels, nerves, the limbs.

In some specific embodiments, the organ is lung, skin, heart, liver, kidney, stomach, intestine, etc.

The complex structure described in the present application is a body structural component composed of different tissues or a body functional component that can perform specific physiological functions or functional activities, such as ears, organs, limbs, eyes, nose, etc.

In a preferred embodiment, the complex structure is an ear hole.

In a specific embodiment, the regeneration and repair is promotion of regeneration after a partial ear resection.

In a specific embodiment, the regeneration and repair is promotion of regeneration and repair after skin damage, hair regeneration after hair loss, regeneration and repair of cartilage and muscle damage, regeneration of lung, liver, skin, heart, kidney, muscle fibrosis, and regeneration after blood vessel, nerve and limb damage.

In a specific embodiment, the regeneration and repair is promotion of the regeneration and repair of scalded skin.

In an embodiment of the present application, the diseases related to the regeneration and repair of tissues and organs include but are not limited to skin scalds, organ fibrosis, muscle/cartilage damage or nervous system diseases, and are preferably skin burns/scalds/traumas, hair loss, cartilage and muscle damage, pulmonary fibrosis, liver fibrosis, kidney fibrosis, myocardial fibrosis, limb trauma or various nervous system diseases.

In a specific embodiment, the composition comprising the retinoic acid receptor activator may also comprise other compounds having regeneration and repair capabilities.

In some specific embodiments, the composition further comprises a protein synthesis inhibitor.

In some specific embodiments, the protein synthesis inhibitor is selected from cycloheximide (CHX), anisomycin (Ani), didemnin B (DIDB), bouvardin (BVD), an Amaryllidaceae plant extract or an Amaryllidaceae alkaloid.

In some specific embodiments, the Amaryllidaceae plant extract or Amaryllidaceae alkaloid comprises Narciclasine or Pancratistatin.

In a specific embodiment, the composition comprises a protein synthesis inhibitor, a retinoic acid receptor activator, and a BMP activator.

In a preferred embodiment, the composition comprises cycloheximide (CHX), all-trans retinoic acid and BMP signaling agonist sb4. Furthermore, in the composition, based on 1 part by weight of cycloheximide, the amount of all-trans retinoic acid is 0.25 to 8 parts by weight and the amount of BMP signaling agonist sb4 is 0.25 to 4 parts by weight.

In another preferred embodiment, the composition comprises Narciclasine, all-trans retinoic acid and BMP signaling agonist sb4. Furthermore, in the composition, based on 1 part by weight of Narciclasine, the amount of all-trans retinoic acid is 0.25 to 10 parts by weight and the amount of BMP signaling agonist sb4 is 2 to 10 parts by weight.

In the embodiments of the present application, pharmaceutically acceptable carriers or excipients may also be added to the medicament or reagent.

Specifically, the medicament or reagent can be prepared in the following form: the protein synthesis inhibitor or a composition comprising the same is mixed with a pharmaceutically acceptable carrier to obtain, for example, oral preparations, such as tablets (including sugar-coated tablets, film-coated tablets, sublingual tablets, orally disintegrating tablets), capsules (including soft capsules, microcapsules), granules, powders, lozenges, syrups, emulsions, suspensions, films (for example, orally disintegrating films), etc., parenteral preparations such as injections (for example, subcutaneous injections, intravenous injections, intramuscular injections, intraperitoneal injections, drops), external preparations (for example, skin preparations, ointments), suppositories (for example, rectal suppositories, vaginal suppositories), pills, nasal drops, respiratory preparations (inhalants), eye drops, etc. Besides, these preparations can be used as controlled release preparations (e.g., sustained release microcapsules), such as immediate release preparations, sustained release preparations and the like. Such preparations can be obtained by preparation methods conventionally used in the art.

Specifically, examples of the above-mentioned pharmaceutically acceptable carriers include excipients (e.g., starch, lactose, sucrose, calcium carbonate, calcium phosphate, etc.), binders (e.g., starch, gum arabic, carboxymethyl cellulose, hydroxypropyl cellulose, crystalline cellulose, alginic acid, gelatin, polyvinyl pyrrolidone, etc.), lubricants (e.g., magnesium stearate, calcium stearate, talc, etc.), disintegrants (e.g., carboxymethyl cellulose calcium, talc, etc.), diluents (e.g., water for injection, saline, etc.), additives (e.g., stabilizers, preservatives, colorants, flavorings, dissolution aids, emulsifiers, buffers, isotonic agents, etc.), and the like.

In an embodiment of the present application, the composition, medicament or reagent may be administered by intraperitoneal injection, intravenous injection, oral gavage, oral administration or skin application. Specifically, the dosage to be administered to a subject varies depending on the administration route, symptoms, patient age, etc., and can be determined practically by a clinician.

### EXAMPLES

Studies in recent years have shown that MRL mice 1 and P 21-/- mutant mice 2 can close 2 mm (millimeter) ear holes, while wild-type experimental mice cannot. Therefore, ear hole closure can be used as a good model for assessing regeneration capacity to screen cells, signaling pathways, and genes that promote and improve regeneration capacity. The main screening targets are important signaling pathways that regulate morphogenesis, organ development, immunity and stress response (metabolism, translation, etc.) during individual development; they also comprise genes and signaling pathways involved in tumorigenesis and regeneration of lower organisms.

Establishment of ear hole trauma mouse model: 7-week-old C57BL/6 mice were selected and anesthetized with 5% chloral hydrate, with 10 mL/kg injected intraperitoneally according to body weight. The anesthetized mice were tied up and the ears and instruments were disinfected with 75% ethanol. The left and right auricles of the mice were punched at the center of the auricle using an 2 mm diameter ear puncher. The traumatized mice were administered drugs (small molecules or growth factors for targeted screening targets dissolved in saline or DMSO) by intraperitoneal injection, intravenous injection or oral gavage, and the solvent without drugs was used as a control. The DMSO-dissolved drug delivery system was: 2-5% DMSO+30-40% PEG400+2-5% Tween 80 were added in sequence according to the final concentration (volume ratio). The drug was administered every 2 days and the mice were observed every 7 days. The proximal-distal diameter (DPD) and anterior-posterior diameter (DAP) of the mouse ear holes were measured using a vernier caliper, and the area of the mouse ear holes at excision was calculated using the area calculation formula S=πDPD×DAP/4. The closure of the mouse ear holes was recorded.

Unless otherwise specified, the ear hole trauma mouse model mentioned above was used in the following Examples of the present application for ear hole regeneration experiments, wherein 7-week-old C57BL/6 mice were purchased from Beijing Charles River Experimental Animal Technology Co., Ltd. ; Nsun2 knockout mice were prepared by the applicant's laboratory, and CRISPR/Cas9-mediated gene knockout technology was used to inject Cas9 mRNA and Nsun2 sgRNA into mouse fertilized eggs to obtain gene knockout embryos, and further breeding obtained stable knockout mice; CHX: (i.e., 4-((R)-2-((1S,3S,5S)-3,5-dimethyl-2-oxocyclohexyl)-2-hydroxyethyl)piperidine-2,6-dione) was purchased from MedCamExpress. Other materials and reagents, unless otherwise specified, can be obtained from commercial sources.

### Example 1: Translation Inhibition (Cycloheximide (Hereinafter Abbreviated as CHX or C); Anisomycin (Hereinafter Abbreviated as Ani)) Promotes the Regeneration at Excision of 2 mm Diameter Mouse Ear Holes

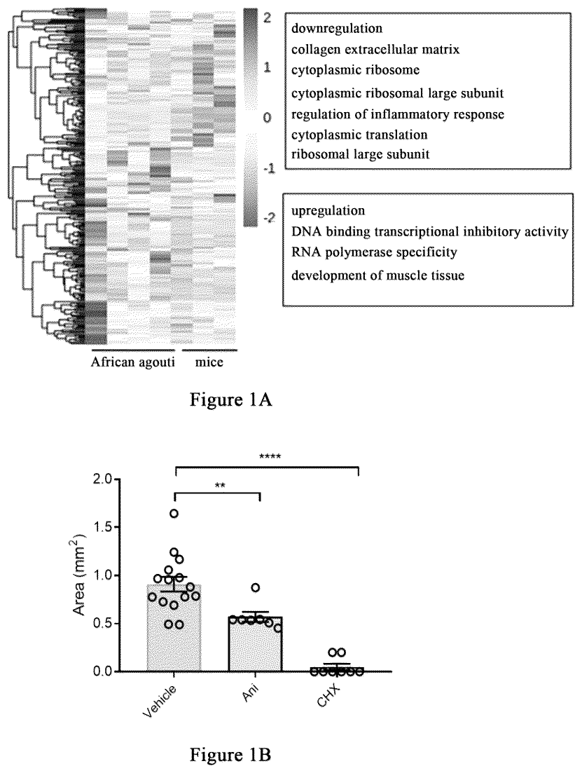

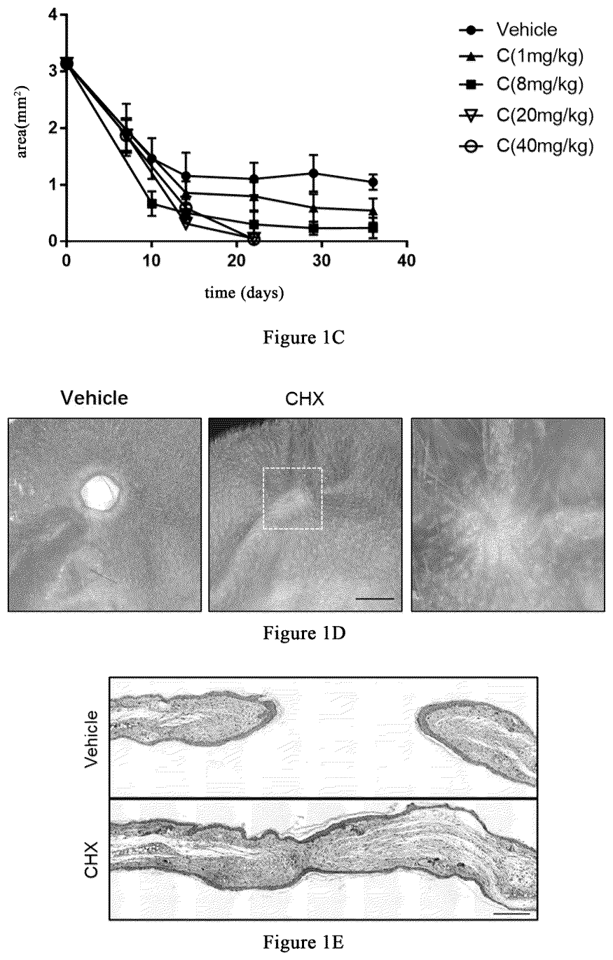

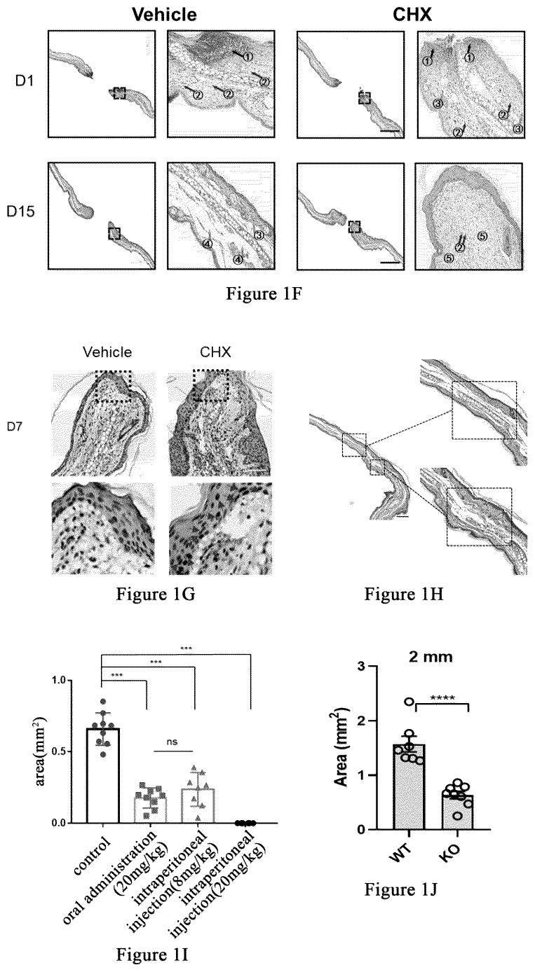

Experimental method: Different doses of CHX (dissolved in saline or DMSO) were intraperitoneally injected into mice with ear hole trauma (in the experiment of FIG. 1I, oral gavage administration was added), and saline or DMSO served as the control group (marked as Vehicle). Identification of traumatic auricles of drug-treated mice was performed. The specific experimental results were shown in FIG. 1.

FIG. 1A showed transcriptome analysis of differentially expressed genes between regenerative African agouti and non-regenerative mice (C57BL/6 mice), and functional enrichment analysis showed that the genes significantly downregulated in regenerative African agouti compared with non-regenerative mice were ribosomal subunits and translation-related.

FIG. 1B showed that based on the analysis results of FIG. 1A, non-regenerative mice were treated with small molecule inhibitors of translation, Ani and CHX, for 21 days respectively, and the healing effect on the 2 mm of ear hole was observed. The results showed that compared with the control group (Vehicle), the translation inhibitors Ani (10 mg/kg) and CHX (20 mg/kg) could significantly promote the healing of the ear hole, and the healing effect of CHX was better. It was found that translation inhibition could significantly promote the regeneration of non-regenerative species. Subsequent experiments were carried out based on CHX.

The schematic diagram of FIG. 1C showed the closure of mouse ear holes after treatment with different concentrations of Vehicle/CHX: each concentration of CHX promoted the closure of a 2 mm diameter of ear holes, and when the concentration was greater than 8 mg/kg, a wound of the mouse ear hole was completely closed after three weeks of drug treatment. n≥8.

FIG. 1D was a photograph showing the healing condition of a 2 mm of ear hole of mice with ear hole trauma treated with Vehicle/CHX (20 mg/kg) 30 days later, in which the ear hole wound treated with CHX had completely closed.

The HE staining image of the mouse auricle tissue in FIG. 1E showed that after treatment with CHX (20 mg/kg), the skin, connective tissue and other tissue structures in the ear hole of the mouse with ear hole trauma grew and connected together, and the trauma had completely healed.

The HE staining image of the mouse auricle tissue in FIG. 1F showed that after the mice with ear hole trauma were treated with DMSO/CHX (20 mg/kg), focal necrosis was locally observed in both tissues on the first day after trauma (D1 group), and inflammatory cells diffusely infiltrated in the necrotic focus, as shown by arrow \textcircled{1}; diffuse infiltration of inflammatory cells was observed in the dermis of the tissue, as shown by arrow \textcircled{2}; among them, hemosiderin deposition was observed in some cells in the CHX group, as shown by arrow \textcircled{3}. On the 15th day, hemosiderin deposition was observed in some cells of the tissues of the DMSO group, as indicated by arrow \textcircled{3}; tissue edema was observed, the spaces between subdermal connective tissue were enlarged, and the tissue structure was loose, as shown by arrow \textcircled{4}. On the 15th day, the CHX group healed the tissue after damage and formed granulation tissue. Fibroblasts and blood vessels proliferated in large numbers in the granulation tissue, as shown by arrow \textcircled{5}. In addition, the inflammatory cells were diffusely distributed, as shown by arrow \textcircled{2}.

The KI67 immunohistochemical staining image of mouse auricle tissue in FIG. 1G showed that after 7 days of CHX (20 mg/kg) treatment, the basal cells of mice expressed a large amount of KI67, a marker protein of cell proliferation, as shown by the arrow, while the expression in the control group was relatively low.

The HE staining image of the mouse auricle tissue in FIG. 1H showed that 180 days after the ear holes of mice treated with CHX (20 mg/kg) were closed, the wound site showed regeneration of tissues and tissue derivatives such as hair follicles, glands, cartilage, muscles and blood vessels.

The schematic diagram of FIG. 1I showed that different administration manners such as oral gavage and intraperitoneal injection were used to treat mice with ear hole trauma, and healing effects were produced after 3 weeks (21 days after damage). Wherein n≥6. ***p<0.001, ns: no significant difference, t-test.

In addition, Nsun2 knockout had been reported to inhibit translation by regulating tRNA stability, so Nsun2 knockout mice were used as a genetic model to verify the effect of translation inhibition on regeneration. The specific method is: ear hole trauma mouse models were established using wild-type mice (WT) and Nsun2 knockout mice (KO), 2 mm diameter of ear holes were made (the method was the same as the above Examples), and the healing condition of the ear holes was observed and measured after 3 weeks. The results showed that the ear hole area of the Nsun 2 knockout mice was significantly reduced compared with that of the wild-type mice. This also suggested that translational inhibition had a role in regeneration (FIG. 1J).

The above experimental results showed that different doses of protein synthesis inhibitor CHX could significantly promote the healing of 2 mm ear holes, and this promoting effect was dose-dependent. Doses greater than 8 mg/kg could promote closure. The closure of the ear hole could regenerate tissue and tissue derivatives such as hair follicles, glands, cartilage and muscle. It was also confirmed that different manners of administration had the effect of promoting regeneration.

### Example 2: Cycloheximide Chx Promotes the Regeneration and Repair of 4 mm Diameter Mouse Ear Hole

The study found that very few mammals, such as the African agouti, have strong regeneration capacities and could regenerate a 4 mm of ear hole. It also found that a closure model of the 4 mm ear hole could distinguish between strong and weak regeneration capacities. Currently, there was no artificial way to achieve closure and regeneration of the 4 mm ear hole. Even the MRL mice reported to have “super regeneration” could not completely close the hole. Therefore, the 4 mm of ear hole could be used as a good model for evaluating the regeneration at excision in mammals. The model was established using the method of Example 1, in which the left and right auricles of mice were punched respectively with a 4 mm diameter ear puncher.

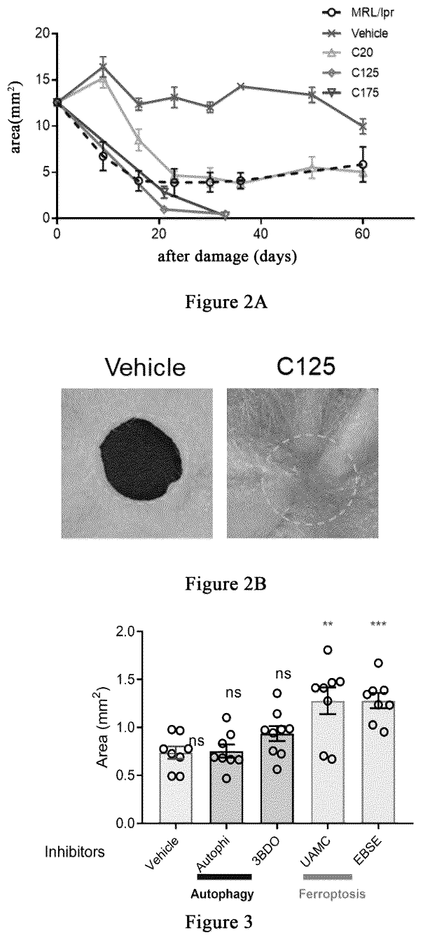

Experimental method: Different doses of CHX (20, 125, 175 mg/kg, dissolved in DMSO or saline) administered by intraperitoneal injection to mice with the 4 mm of ear hole trauma. DMSO served as the control group. Identification of traumatic auricles of drug-treated mice was performed. The specific experimental results were shown in FIG. 2.

The schematic diagram of FIG. 2A showed the effect of different doses of CHX on the closure of 4 mm diameter of ear holes. It was found that 20 mg/kg had a similar effect to that of super-healing mice MRL/lpr, which promoted the shrinkage of ear hole wounds, but could not completely close them. When the dose was greater than 20 mg/kg (125, 175 mg/kg), complete closure of the ear holes could be achieved.

FIG. 2B was a photograph of healing of the ear hole after treatment with DMSO/CHX drug for 90 days, which shows that the wound area of the 4 mm mouse ear hole treated with CHX (125 mg/kg) was significantly reduced.

### Example 3: Cycloheximide (CHX) Promotes Ear Hole Regeneration Independently of the Inhibition of Ferroptosis and Autophagy

As an antifungal antibiotic, CHX could inhibit ferroptosis and cellular autophagy in addition to inhibiting eukaryotic protein synthesis and RNA synthesis. In order to verify which target it acts through during the regeneration process, small molecule validation was performed targeting ferroptosis and autophagy respectively.

Experimental methods: The same ear hole trauma mouse model was used for the experiment, and inhibitors of ferroptosis and autophagy were used instead of CHX to observe their effects on promoting the healing of the ear hole. The control group in which DMSO replaced the small molecule. Auto: Autophinib (MCE, HY-101920), inhibits autophagy; 3BDO: 3BDO (MCE, HY-U00434), inhibits autophagy; UAMC: UAMC-3203 (MCE, MCE, HY-112909A), inhibits ferroptosis; EBSE: Ebselen (MCE, HY-13750), inhibits ferroptosis. The concentration used was 10-20 mg/kg, and the administration manner was the same as CHX, n≥8, **p<0.01, ***p<0.001, ns: no significant difference, t-test. Identification of the effects of different inhibitors on the healing of 2 mm ear holes was performed (measured 21 days after damage). The results were shown in FIG. 3: Neither inhibitors of ferroptosis nor autophagy could promote the healing of the ear hole like CHX, which indicates that CHX's promotion of ear hole regeneration was not dependent on its inhibitory activity on ferroptosis or autophagy.

### Example 4: Inhibition of Rars Inhibits CHX Regeneration

To further verify the signaling pathways that play a role in CHX-promoting ear hole regeneration, screening was performed by simultaneously interfering with developmental and lower organism regeneration-related pathways while adding CHX.

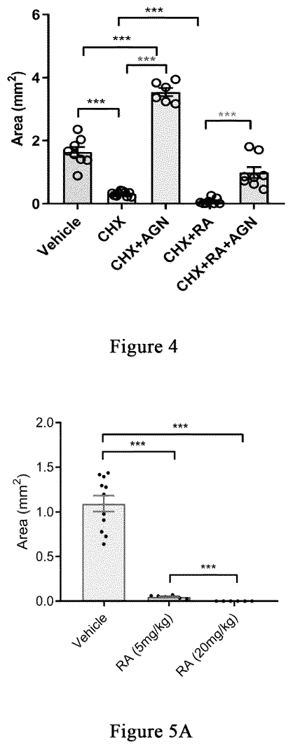

Experimental method: CHX (20 mg/kg), CHX+AGN, CHX+RA, and CHX+RA+AGN were intraperitoneally injected into mice with 2 mm ear hole trauma respectively. Among them, AGN was AGN 193109 (AGN, MCE, HY-U 00449, 0.75 mg/kg), a retinoic acid receptors (RAR) specific inhibitor, which antagonizes RARs; RA was all-trans retinoic acid (ATRA, MCE, HY-14649, 5 mg/kg), a RAR activator, which activates RARs. DMSO was used as the control group. n≥6. ***p<0.001, t-test. 21 days after damage, the trauma area of the ear holes of the drug-treated mice in each group was measured. The specific experimental results were shown in FIG. 4.

The results showed that RAR pathway inhibitors and activators had inhibitory and synergistic effects on CHX, respectively. Among them, AGN193109, a retinoic acid receptors (RAR) specific inhibitor, could completely inhibit the healing of the ear holes induced by CHX. The combined use of CHX and the all-trans retinoic acid, a RAR activator, could produce a synergistic effect in promoting the closure of the ear hole, and this coordinated effect could still be inhibited by the AGN, a RAR inhibitor. This suggested that RARs might play an important role in the ear hole regeneration model.

### Example 5: Activating RARγ To Promote Ear Hole Regeneration

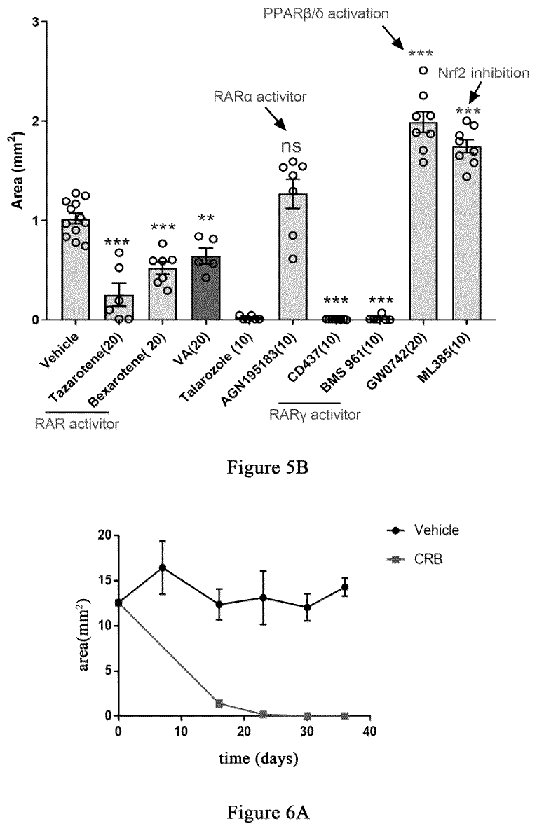

To further verify whether direct activation of RARs could play a role similar to CHX in promoting ear hole regeneration, ear hole trauma experiments were performed with DMSO/RA (5 mg/kg, 20 mg/kg). The results of FIG. 5A (21 days after ear hole damage) showed that RA alone could also promote the closure of the ear hole in a dose-dependent manner.

Since RA could equally activate the three subtypes of RA receptors: RARα, RARβ, and RARγ, and could also act on and activate PPARβ/δ6, and inhibit the transcription factor Nrf27, in order to further confirm the specific targets on which it acts to promote ear hole regeneration, each potential target was interfered with for verification. The experimental method: the following reagents were administered to the ear hole trauma mouse model, and the healing of the ear hole was detected 21 days after the trauma.

Tazarotene (MCE, HY-15388, 20 mg/kg) and Bexarotene (MCE, HY-14171, 20 mg/kg), retinoic acid receptor agonists; VA (VitaminA, Selleck, S5592, 20 mg/kg), vitamin A, metabolized in vivo to produce RA; Talarozole (MCE, HY-14531, 10 mg/kg), inhibited CYP26A1 and CYP26B1, and increased endogenous all-trans retinoic acid; AGN 195183 (MCE, HY-16684, 10 mg/kg), a selective RARα agonist; CD437 (MCE, HY-100532, 10 mg/kg) and BMS 961 (Glpbio, GC17382, 10 mg/kg), a selective RARy agonist; GW0742 (MCE, HY-13928, 20 mg/mg), activated PPARβ/δ; ML385 (MCE, HY-100523, 10 mg/kg), a specific Nrf2 inhibitor. n≥5, **p<0.01, ***p<0.001, ns: no significant difference, t test. The experimental results were shown in FIG. 5B.

The results showed that activation of RARγ (including RAR activator and RARγ selective activator) could promote ear hole regeneration, while activation of RARα had no significant promoting effect. Activation of PPARβ/δ and inhibition of Nrf2 inhibited the healing at the basal level. In addition, increasing endogenous RA levels by supplementing vitamin A or using Talarozole, an all-trans retinoic acid metabolism blocker, could also promote ear hole regeneration, which enriched the regulatory targets for activating RARs.

### Example 6: The Combination of CRB (C: Protein Synthesis Inhibitor CHX; R: RAR Activator All-Trans Retinoic Acid; B: BMP Activator BMP (Signaling Agonist sb4)) Promotes The Closure of 4 mm Mouse Ear Holes and Regeneration at Excisions

Taking 7-week-old mice as an example, the mice were anesthetized with 5% chloral hydrate, with 10 mL/kg injected intraperitoneally according to body weight. The anesthetized mice were tied up and the ears and instruments were disinfected with 75% ethanol. The left and right auricles of the mouse were punched at the center of the auricle using a 4 mm diameter ear puncher. For mice after trauma, DMSO/CRB (The dosage of CRB was 20 mg/kg CHX, 20 mg/kg total ATRA, 10-20 mg/kg BMP signaling agonist sb4) was intraperitoneally injected once every 2 days, and the mice were anesthetized every 7 days. The proximal-distal diameter (DPD) and anterior-posterior diameter (DAP) of the mouse ear holes were measured using a vernier caliper, and the area of the mouse ear holes at excision was calculated. The area calculation formula was S=π×DPD×DAP/4. The traumatic auricles of drug-treated mice were further identified.

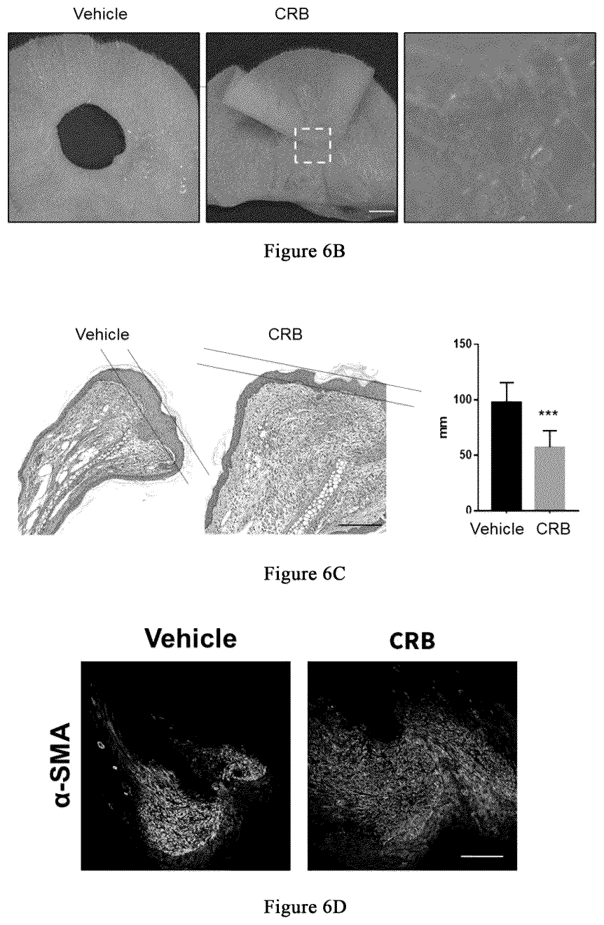

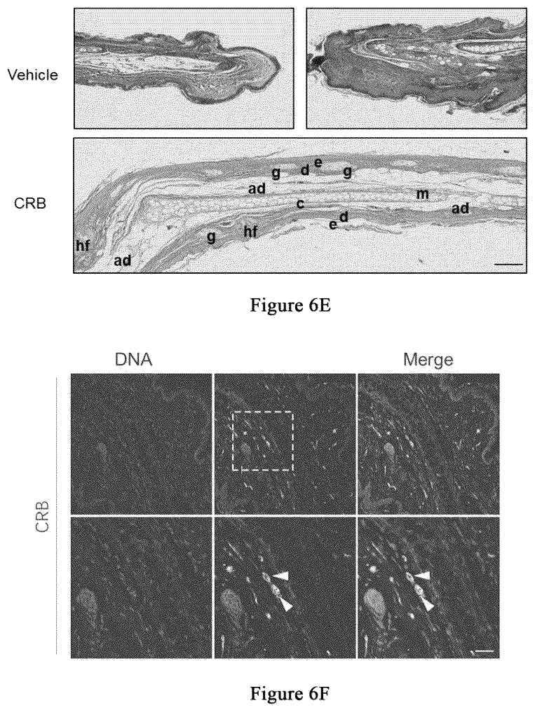

FIG. 6A showed the closure of the mouse ear holes after treatment with DMSO/CRB respectively. The results showed that CRB promoted the closure of a 4 mm diameter ear holes. After 30 days of treatment, the mouse ear hole wound were completely closed.

FIG. 6B showed the closure of the 4 mm mouse ear holes after 30 days of drug treatment. It could be seen that the ear hole trauma of the mice treated with drugs had closed.

FIG. 6C showed the HE staining results and epidermal thickness statistics of mouse auricle tissue after 7 days of drug treatment. The scale bar was 200 μm. After drug treatment, blastema was formed in the auricle of mice and the thickness of the epidermis was significantly reduced. n≥3, t-test.

FIG. 6D showed the results of α-SMA immunofluorescence staining in mouse auricle tissue after 7 days of drug treatment. The scale bar was 100 μm. Seven days after trauma, both the control group and the drug-treated group showed a large amount of α-SMA expression. Compared with the control group, the α-SMA expression in the drug-treated group was linearly arranged in an orderly manner, while the control group was disorderly accumulated. The expression pattern was similar to the scar formation manner.

FIG. 6E showed the regeneration of various tissues after drug (CRB) induction for more than 90 days, showing the regeneration of epidermis, dermis, glands, hair follicles, muscle, cartilage, fat, and muscle. “e” indicated epidermis regeneration; “d” indicated regenerated dermis; “g” indicated regenerated gland; “ad” indicated regenerated adipose tissue; “hf” indicated regenerated hair follicle; “c” indicated regenerated cartilage; and “m” indicated regenerated muscle.

FIG. 6F further identified angiogenesis by immunofluorescence staining of the vascular marker CD31, wherein triangles indicated regenerated blood vessels.

### Example 7: RA Reduces Scar Formation After Scalds in Mice and Promotes Skin Regeneration

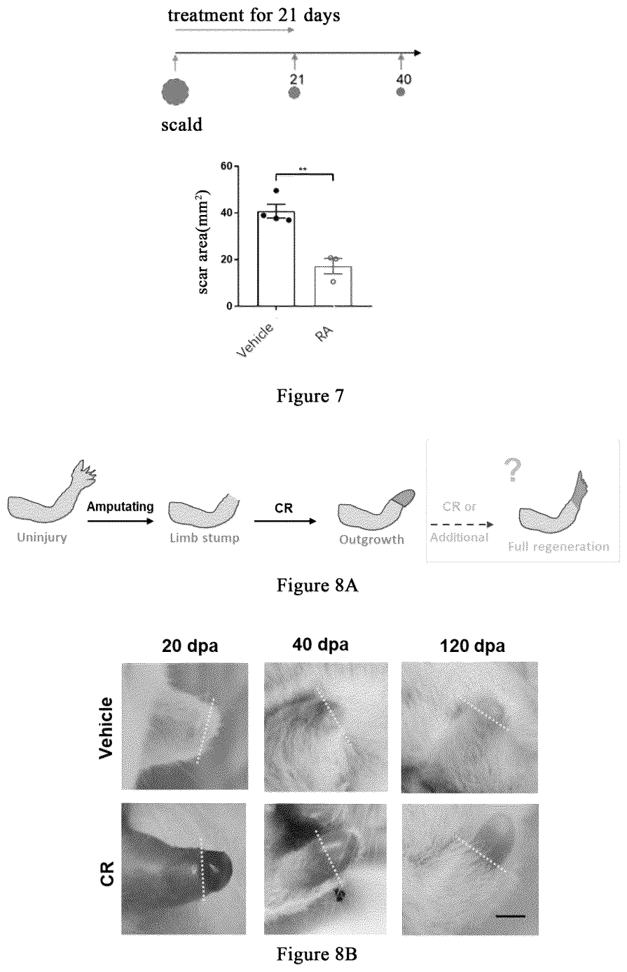

Experimental method: 7-week-old mice were used and (1) mice were anesthetized by intraperitoneal injection of 0.5% pentobarbital sodium (100 μl/10 g b.w.); (2) the back was shaved, and the skin was disinfected with 75% alcohol. (3) the mice were scalded with boiling water for 10-15 seconds. The scald area was a circle with a diameter of 1.5 cm. (4) After scalding, the mice were divided into two groups and treated with Vehicle (DMSO) and CHX (20 mg/kg, dissolved in DMSO), respectively. (5) wound repair status was detected. The experimental results were shown in FIG. 7.

Results: After 40 days of administration, the scar test results after hair shaving showed that RA treatment could significantly reduce the scar area.

### Example 8: Optimized Combination of Cycloheximide CHX (C) and All-Trans Retinoic Acid (R) Promotes Regeneration and Repair of Mouse Limb Outgrowth Structure

Based on the regeneration effect of ear hole, we next conducted regeneration induction experiments with more complex damage phenotypes, using limb resection as the damage model. ICR mice were subjected to limb modeling, and appropriate doses of CHX (100 mg/kg) and RAR (retinoic acid receptor) activator, all-trans retinoic acid (ATRA, 20 mg/kg) were combined and intraperitoneally administered every other day for 8 weeks to observe the regenerative phenotype.

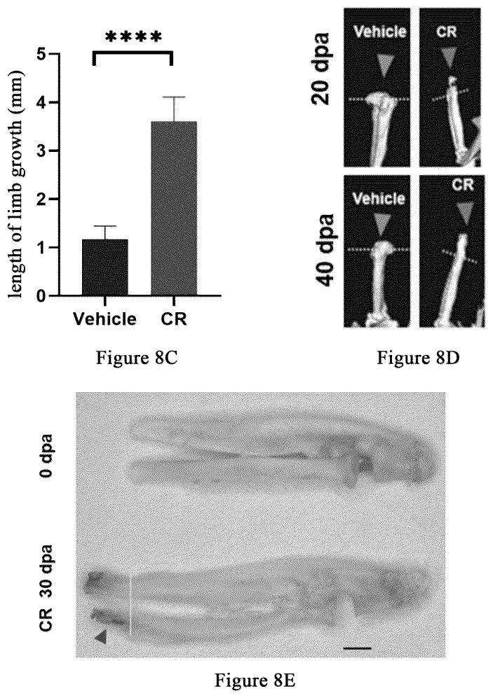

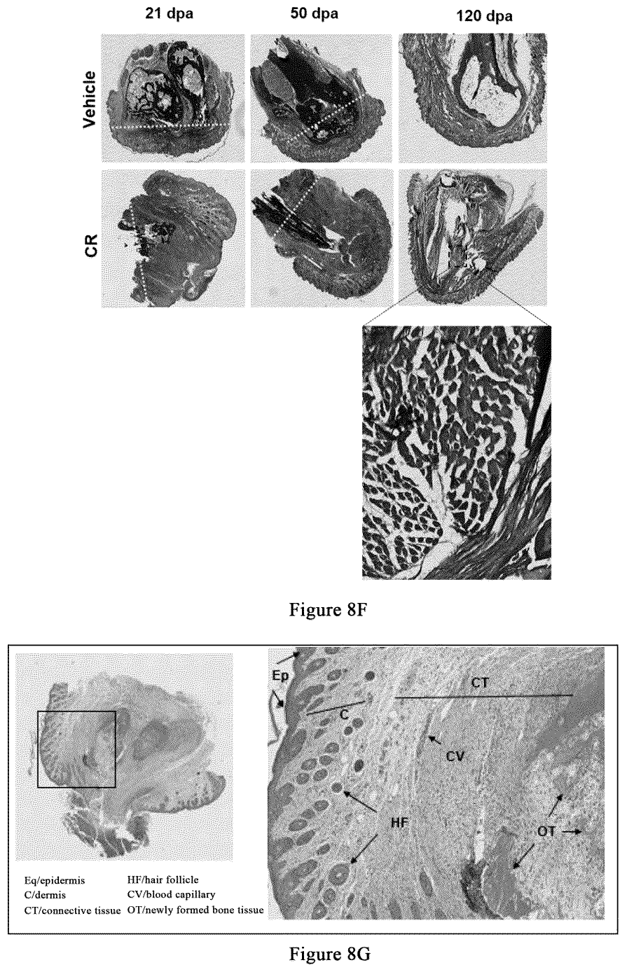

Experimental method: 8-week-old ICR mice were used as the model. Mice were anesthetized with 5% chloral hydrate, with 10 mL/kg injected intraperitoneally according to body weight. The anesthetized mouse was tied up and the left upper limb of the mouse and surgical instruments were disinfected with 75% ethanol. In order to accurately define the occurrence of regeneration, the distance from the elbow joint to the radius and ulna was measured, and 10 mm was retained. The rest to the palm, fingers and other segments were resected for modeling (the ulna and radius were two bones of the forearm. The easiest way to distinguish them was that the thumb side was the radius and the little finger side was the ulna). CHX (100 mg/kg, dissolved in DMSO or saline) and ATRA (20 mg/kg) were intraperitoneally injected every other day for 8 weeks. DMSO served as the control group, and the phenotype was continuously observed. After different days, the limb traumas of drug-treated mice were identified. The specific experimental results were shown in FIG. 8. FIG. 8A was a schematic diagram of limb modeling. FIG. 8B showed the phenomenon of limb regeneration induced by drug treatment 20, 40, and 120 days later. It could be seen that outgrowth occurred at the amputation site of the mice treated with the drug. Scale bar was 2 mm. FIG. 8C was the statistical result of the limb outgrowth length. It could be seen that the CR-treated group induced limb regeneration very well, and the length was significantly different from that of the control group. n≥3, ***p<0.001, t-test. FIG. 8D showed high-definition images of the bone tissue reconstruction process obtained by electronic computed tomography (CT) after 20 and 40 days of drug CR treatment. The CR-treated group showed a good induced growth process of limb bone tissue, and elongated reconstructed fragments of bone tissue could be observed 20 days after CR treatment (right figure, indicated by triangle arrows), while the control group showed a blunt state, showing hyperplasia and accumulation of bone tissue, and did not show a forward growth trend (left figure, indicated by triangle arrows), which was a similar occurrence to scar repair. FIG. 8E was a more direct and critical evidence of bone regeneration. After 30 days of drug CR treatment, the complete radius and ulna were taken out for observation. The results were consistent with the CT scan results, and the regeneration trend of bone tissue could be clearly seen. In addition, HE section staining revealed that the combination could promote the regeneration of multiple tissue types in the amputated limb, such as skin, hair follicles, capillaries, new bone and other tissues (FIG. 8F, “Ep” indicated epidermal regeneration; “CT” indicated connective tissue regeneration; “CV” indicated capillary regeneration; “HF” indicated hair follicle regeneration; “OT” indicated bone tissue regeneration.). FIG. 8G showed the results of Masson staining of mouse limb regeneration tissue after 21, 50, and 120 days of drug treatment. The blunt morphology of bone tissue was observed in the control group, which was consistent with the CT results. The bone tissue in the drug-treated group showed a growth trend. Importantly, regeneration of muscle tissue was observed in the experimental group at 120 days, while the control group showed disordered accumulation, and the expression pattern was similar to scar formation. Scale bar was 2 mm, n≥3, t-test.

The above results show that the composition of cycloheximide CHX and all-trans retinoic acid (ATRA) could promote the regeneration and repair of the outgrowth structure after limb resection, including the regeneration of bones, muscles, skin, hair follicles, capillaries, connective tissue, new bone tissue, etc.

### Example 9: The Combination of NRB (N: Narciclasine; R: RAR Activator All-Trans Retinoic Acid; B: BMP Activator BMP (Signaling Agonist sb4)) Promotes the Regeneration of a 4 mm Mouse Ear Hole

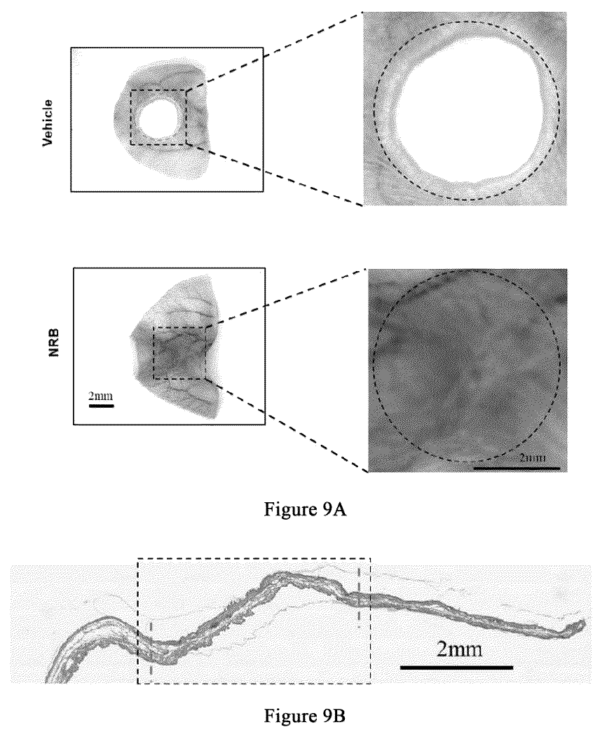

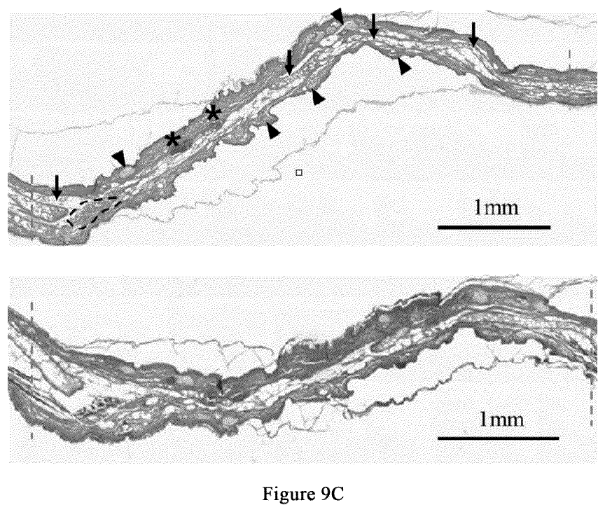

Experimental method: The method for constructing the mouse model was the same as in Example 2. Mice with 4 mm ear hole trauma were intraperitoneally injected with DMSO/NRB (NRB: Narciclasine 3 mg/kg, ATRA 20 mg/kg, BMP signaling agonist sb4 10-20 mg/kg) once every 2 days, and the mice were anesthetized every 7 days. The proximal-distal diameter (DPD) and anterior-posterior diameter (DAP) of the mouse ear holes were measured using a vernier caliper, and the area of the mouse ear holes at excision was calculated. The area calculation formula was S=π×DPD×DAP/4. The traumatic auricles of drug-treated mice were further identified. The experimental results were shown in FIGS. 9A to 9C.

Among them, FIG. 9A showed the healing effect of the 4 mm mouse ear hole after 30 days of NRB treatment, and showed that it had a healing-promoting effect. After 30 days of drug treatment, the wound of the mouse ear hole was completely closed, which was identified as a regeneration event. FIGS. 9B and 9C showed that the HE section staining data well indicating the regeneration structures of structures such as cartilage (long black arrows), hair follicles (asterisks), glands/sebaceous glands (triangular arrows), and muscles (dashed line boxed areas).

The above experimental results showed that the NRB small molecule composition could also promote the closure of 4 mm ear holes and promote regeneration.

The foregoing merely illustrates the principles of the present invention, and it should be understood that the scope of the present invention is not intended to be limited to the exemplary aspects described herein, but rather to encompass all currently known and future developed equivalents. In addition, it should be pointed out that several improvements and modifications may be made without departing from the technical principles of the present invention, and these improvements and modifications should also be regarded as within the scope of the present invention.

1. Use of a retinoic acid receptor activator or a composition comprising the retinoic acid receptor activator in the promotion of regeneration and repair capacity of a tissue, complex structure or organ of a mammal or in the treatment of a disease related to regeneration and repair of a tissue, complex structure or organ of a mammal.

2. (canceled)

3. (canceled)

4. The use according to claim 1, wherein the retinoic acid receptor activator comprises any one or more selected from the group consisting of: an all-trans retinoic acid and a selective retinoic acid receptor γ agonist, preferably the selective retinoic acid receptor γ agonist is CD437 and/or BMS961.

5. The use according to claim 1, wherein the regeneration and repair is promotion of regeneration of a tissue, complex structure or organ after tissue or organ resection or damage.

6. The use according to claim 1, wherein
the tissue is skin, fat, muscle, bone, hair follicle, blood vessel, or nerve;
the complex structure is at least two or more of body structure comprising skin, hair follicle, gland, cartilage, muscle, fat, blood vessel, nerve, or limb; and
the organ is lung, liver, heart, pancreatic islet, or kidney.

7. The use according to claim 1, wherein the complex structure is an ear, a limb, a finger, an eye, or a nose.

8. The use according to claim 1, wherein the regeneration and repair is promotion of regeneration and repair after skin damage, hair regeneration after hair loss, regeneration and repair of cartilage and muscle damage, regeneration of fibrosis of lung, liver, skin, heart, kidney and muscle, and regeneration after blood vessel, nerve and limb damage.

9. The use according to claim 1, wherein the disease is skin scald, skin trauma, skin burn, hair loss, cartilage and muscle damage, liver fibrosis, pulmonary fibrosis, or limb damage.

10. The use according to claim 1, wherein the promotion of regeneration and repair of the tissue, complex structure or organ of the mammal is achieved by activating retinoic acid, preferably by activating retinoic acid receptor γ.

11. A composition, wherein it comprises a protein synthesis inhibitor, and an all-trans retinoic acid and a BMP activator, preferably the protein synthesis inhibitor is Cycloheximide (CHX) or Narciclasine, and the BMP activator is BMP signaling agonist sb4.

12. The composition according to claim 11, wherein in the composition, based on 1 part by weight of the cycloheximide, the amount of the all-trans retinoic acid is 0.25 to 8 parts by weight, and the amount of the BMP signaling agonist sb4 is 0.25 to 4 parts by weight.

13. The composition according to claim 11, wherein in the composition, based on 1 part by weight of the Narciclasine, the amount of the all-trans retinoic acid is 0.25 to 10 parts by weight, and the amount of the BMP signaling agonist sb4 is 2 to 10 parts by weight.

14. The composition according to claim 11, wherein the composition is administered by intraperitoneal injection, intravenous injection, oral gavage, oral administration, or skin application.

15. The use according to claim 1, wherein the composition is a composition comprising a protein synthesis inhibitor, and an all-trans retinoic acid and a BMP activator, preferably the protein synthesis inhibitor is Cycloheximide (CHX) or Narciclasine, and the BMP activator is BMP signaling agonist sb4.

16. A method for promoting regeneration and repair of a tissue, complex structure or organ of a mammal, comprising administering a retinoic acid receptor activator or a composition comprising the retinoic acid receptor activator to a subject in need thereof.

17. The method according to claim 16, wherein the retinoic acid receptor activator comprises any one or more selected from the group consisting of: an all-trans retinoic acid and a selective retinoic acid receptor γ agonist, preferably the selective retinoic acid receptor y agonist is CD437 and/or BMS961.

18. The method according to claim 14, wherein the composition comprising the retinoic acid receptor activator is a composition comprising a protein synthesis inhibitor, and an all-trans retinoic acid and a BMP activator, preferably the protein synthesis inhibitor is Cycloheximide (CHX) or Narciclasine, and the BMP activator is BMP signaling agonist sb4.
